(19) United States
(12) Patent Application Publication
(10) Pub. No.: US 2023/0211790 A1
(43) Pub. Date: Jul. 6, 2023

(54) MULTI-FUNCTION INPUT DEVICES FOR VEHICLES

(71) Applicants: Hyundai Motor Company, Seoul (KR); Kia Corporation, Seoul (KR)

(72) Inventor: Brian Phelps, Ypsilanti, MI (US)

(21) Appl. No.: 17/569,036

(22) Filed: Jan. 5, 2022

(51) Int. Cl. B60W 40/09; B60W 50/10; B62D 15/02; H04W 4/46

(52) U.S. Cl. CPC B60W 40/09; B60W 50/10; B62D 15/02; H04W 4/46

(57) ABSTRACT

Methods and systems are described for executing vehicle functions. The system includes a sensor, an input device, and a controller that is configured to adjust a plurality of vehicle functions based on user identification. The controller detects a first user input received at the input device and identifies a first user based on a first signal received from the sensor, the first signal corresponding to a first vehicle function. The controller adjusts the first vehicle function corresponding to the first signal and detects a second user input received at the input device. Additionally, the controller identifies a second user based on a second signal received from the sensor, the second signal corresponding to a second vehicle function. The controller adjusts the second vehicle function corresponding to the second signal. The first vehicle function and the second vehicle function perform different vehicle functions.

### TECHNICAL FIELD

The present disclosure relates generally to multi-function input devices, and more particularly, to multi-function input devices for vehicles.

### BACKGROUND

Generally, vehicles carry multiple occupants therein to a destination. As is well known, vehicles are equipped with user interfaces for adjusting various vehicle functions. Some vehicles allow occupants to customize the user interfaces or input devices within a vehicle to make the user interface more familiar and convenient. However, a customized input device may frustrate other occupants or vehicle users who are unfamiliar with the user interface or the customized input device. In other words, other passengers within the vehicle or other vehicle users in shared vehicles may be unfamiliar with the particular customized user interface. Other occupants may have particular personal preferences in making the vehicle user interface familiar and convenient. However, currently vehicle occupants must experiment with input devices to determine their functionality, especially with unlabeled input devices. More worrisome, occupants are unable to adapt previously customized input devices in the vehicle to satisfy their own preferences.

### SUMMARY

The present disclosure provides methods, systems, articles of manufacture, including computer program products, for multi-function input devices for vehicles.

In one aspect, a system for executing vehicle functions is provided. The system includes a sensor mounted on a vehicle and an input device communicatively coupled to the sensor that is configured to adjust a plurality of vehicle functions based on user identification. The system includes a controller communicatively coupled to the sensor and the input device, the controller is configured to detect a first user input received at the input device and identify a first user based on a first signal received from the sensor, the first signal corresponding to a first vehicle function. In response to detecting the first user input received at the input device, the controller adjusts the first vehicle function corresponding to the first signal and detects a second user input received at the input device. The controller identifies a second user based on a second signal received from the sensor, the second signal corresponding to a second vehicle function. In response to detecting the second user input received at the input device, the controller adjusts the second vehicle function corresponding to the second signal, where the first vehicle function and the second vehicle function perform different vehicle functions.

In some variations, one or more of the features disclosed herein including the following features may optionally be included in any feasible combination. In some variations, wherein the sensor is a fingerprint reader and the sensor is integrated into the input device. In some variations, the first user maps the input device to the first vehicle function, the second user maps the input device to the second vehicle function, and the input device is at least one of a tactile button and a capacitive button. In some variations, adjusting the first vehicle function is based on the first user inputting the first user input at the input device and wherein adjusting the second vehicle function is based on the second user inputting the second user input at the input device.

Additionally, the controller may be further configured to identify, based on the sensor, the first user in response to detecting the first user input at the input device; and identify, based on the sensor, the second user in response to detecting the second user input at the input device. In some variations, the input device and the sensor are integrated into at least one of a steering wheel and a console of the vehicle. In some variations, the first vehicle function and the second vehicle function are associated with a subset of the plurality of vehicle functions.

Further, the system may further include an additional sensor and the controller is configured to adjust the first vehicle function based on a first data reading from the additional sensor; and adjust the second vehicle function based on a second data reading from the additional sensor, wherein the first data reading corresponds to a first user biometric and the second data reading corresponds to a second user biometric. In some variations, the additional sensor is a temperature sensor, the first data reading is a temperature reading greater than a predetermined temperature and the second data reading is the temperature reading less than the predetermined temperature.

Additionally, in some variations, adjusting the first vehicle function at the vehicle includes at least one of decreasing a steering wheel temperature and decreasing an air temperature in the vehicle based on the temperature reading being greater than the predetermined temperature, and wherein adjusting the second vehicle function is at least one of increasing the steering wheel temperature and increasing the air temperature in the vehicle based on the temperature reading being less than the predetermined temperature. In some variations, the additional sensor is a heart rate sensor, the first data reading is a heart rate reading greater than a predetermined rate and the second data reading is the heart rate reading less than the predetermined rate. Adjusting the first vehicle function includes at least one of diffusing a calming fragrance and selecting calming music based on the heart rate reading being greater than the predetermined rate, and wherein adjusting the second vehicle function includes at least one of alerting passengers in the vehicle and contacting emergency services based on the heart rate reading being less than the predetermined rate.

Implementations of the current subject matter may include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which may include a non-transitory computer-readable or machine-readable storage medium, may include, encode, store, or the like one or more programs that cause one or more processors to perform one or more of the operations described herein. Computer-implemented methods consistent with one or more implementations of the current subject matter may be implemented by one or more data processors residing in a single computing system or multiple computing systems.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow this disclosure are intended to define the scope of the protected subject matter.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1A depicts an example of a diagram illustrating a first user selecting an input device associated with a first vehicle function based on detecting the first user;

FIG. 1B depicts an example of a diagram illustrating a second user selecting a input device associated with a second vehicle function based on detecting the second user;

FIG. 2 depicts an example of a flowchart for adjusting a first vehicle function or a second vehicle function at a vehicle based on user identification;

FIG. 3A depicts an example of a user interface for assigning an input device to a first vehicle function based on identifying a user;

FIG. 3B depicts an example of a user interface for assigning an input device to a first vehicle function based on identifying a user;

FIG. 4A depicts an example of a figure illustrating a temperature sensor;

FIG. 4B depicts an example of a figure illustrating a heart rate sensor;

FIG. 4C depicts an example of a figure illustrating a capacitive input device;

FIG. 5 depicts an example of another flowchart for adjusting a first vehicle function or a second vehicle function at a vehicle based on user identification and at least one of a temperature sensor, a heart rate sensor, and a capacitive input device;

FIG. 6 depicts an example of a figure illustrating the various places for the input device in a vehicle;

FIG. 7 depicts an example of a figure illustrating the various places for the input device on a steering wheel;

FIG. 8 depicts an example of a figure illustrating the various places for the input device in a console; and

FIG. 9 depicts a block diagram illustrating a computing system consistent with implementations of the current subject matter.

### DETAILED DESCRIPTION

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

Although exemplary embodiments are described as using a plurality of units to perform the exemplary process, it is understood that the exemplary processes may also be performed by one or plurality of modules. Additionally, it is understood that the term controller/control unit refers to a hardware device that includes a memory and a processor. The memory is configured to store the modules and the processor is specifically configured to execute said modules to perform one or more processes which are described further below.

Furthermore, control logic of the present embodiments may be embodied as non-transitory computer readable media on a computer readable medium containing executable program instructions executed by a processor, controller/control unit or the like. Examples of the computer readable mediums include, but are not limited to, ROM, RAM, compact disc (CD)-ROMs, magnetic tapes, floppy disks, flash drives, smart cards and optical data storage devices. The computer readable recording medium may also be distributed in network coupled computer systems so that the computer readable media is stored and executed in a distributed fashion, e.g., by a telematics server or a Controller Area Network (CAN).

The terminology used herein is for the purpose of describing particular embodiments only and is not intended to be limiting of the embodiments. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” may be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The present disclosure provides a system for adjusting different vehicle functions associated with an input device based on identifying a vehicle occupant. The system may be configured to detect a first user input received at the input device. The system may be configured to identify a first user based on a first signal received from the sensor. For example, a vehicle occupant may press or otherwise engage a button with a fingerprint reader that identifies the vehicle occupant. The first signal may correspond to a vehicle function. The system may be configured to adjust the vehicle function corresponding to the first signal in response to detecting the first user input received at the input device. For example, pressing the button and the fingerprint of the occupant may be mapped to the vehicle functionality of adjusting the climate control to a preferred setting.

Then, the system may be configured to detect a second user input received at the input device. The system may be configured to identify a second user based on a second signal received from the sensor. For example, a second vehicle occupant may press the same button with a fingerprint reader that identifies the second vehicle occupant. The second signal may correspond to a second vehicle function. The system may be configured to adjust the second vehicle function corresponding to the second signal in response to detecting the second user input received at the input device. For example, pressing the button and the fingerprint of the second occupant may be mapped to the vehicle functionality of adjusting the stereo system to a preferred setting. The first vehicle function and the second vehicle function may perform different vehicle functions.

The input device may be a tactile button or a capacitive button. The input device may be integrated into a steering wheel or a console of the vehicle. The controller may be configured to adjust the first vehicle function based on the first user inputting the first user input at the input device. The controller may be configured to adjust the second vehicle function based on the second user inputting the second user input at the input device. In some embodiments, the controller and the sensor may be configured to identify the first user in response to detecting the first user input at the input device. Similarly, the sensor may be configured to identify the second user in response to detecting the second user input at the input device.

Additionally, the system may include an additional sensor such as a temperature sensor or a heart rate monitor. The controller may be configured to adjust the first vehicle function or the second vehicle function based on data readings from the additional sensor. Data readings may correspond to a first user biometric or a second user biometric. For example, data readings from a finger temperature reading greater than a predetermined temperature or a finger temperature reading lower than the predetermined temperature. In response, the first vehicle function may be adjusted by decreasing a steering wheel temperature or decreasing an air temperature in the vehicle based on the finger temperature reading being greater than the predetermined temperature. Alternatively, the vehicle function may be adjusted by increasing the steering wheel temperature or increasing the air temperature in the vehicle based on the fingerprint temperature reading being less than the predetermined temperature.

In some embodiments, the system may include an additional sensor such as a heart rate sensor configured to detect whether a heart rate reading is greater than a predetermined rate or less than the predetermined rate. The controller may be configured to adjust the first vehicle function to diffuse a calming fragrance or select calming music based on the heart rate reading being greater than the predetermined rate. Additionally, the controller may be configured to adjust the second vehicle function to alert passengers in the vehicle and contact emergency services based on the heart rate reading being lower than the predetermined rate.

The methods, systems, apparatuses, and non-transitory storage mediums described herein for adjusting different vehicle functions associated with an input device based on identifying a vehicle occupant. The various embodiments also utilize an additional sensor such as a temperature sensor or a heart rate monitor for further adjusting vehicle functionalities.

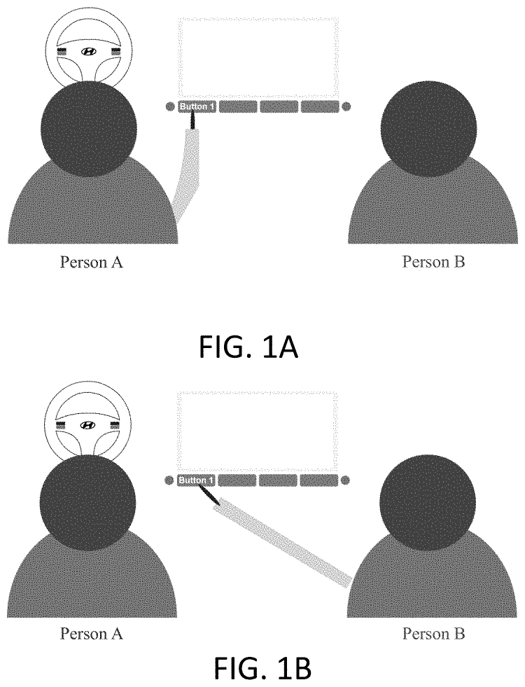

Referring now to the corresponding drawings, FIG. 1A depicts an example of a diagram illustrating a first user selecting an input device associated with a first vehicle function based on detecting the first user. The input device may be a button at the head console. The input device may correspond to a vehicle functionality upon identifying a user and detecting input at the input device. For example, the button may adjust the climate control system in response to identifying the first user and the input at the button.

FIG. 1B depicts an example of a diagram illustrating a second user selecting a input device associated with a second vehicle function based on detecting the second user. The input device may be a button at the head console. The input device may correspond to a second vehicle functionality in response to identifying a second user and detecting input at the input device. For example, the button may engage a hands-free Bluetooth mode in response to identifying the second user and the input at the button.

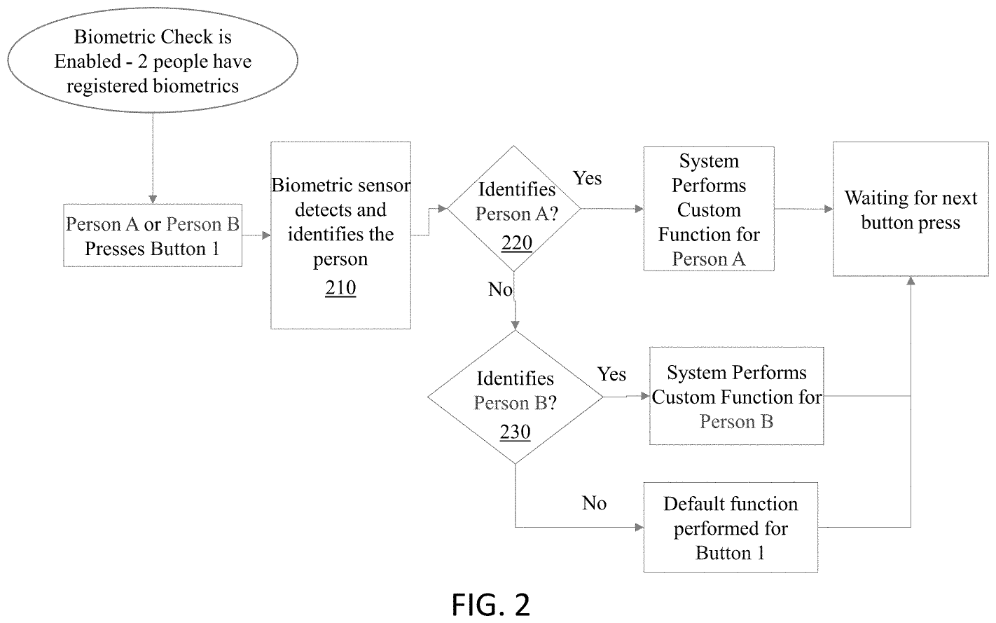

FIG. 2 depicts an example of a flowchart for adjusting a first vehicle function or a second vehicle function at a vehicle based on user identification. A controller may be configured to determine user identification and then adjust the vehicle function based on the user identification and input at an input device. The controller may be communicatively coupled to a sensor, a button, a temperature sensor, a heart rate monitor, and a capacitive button. The controller may be configured to detect data readings from the sensor, the input device, the button, the temperature sensor, the heart rate monitor, and the capacitive button. However, the present disclosure is not limited to these devices.

At 210, the controller may be configured to receive a data reading to identify a person based on the biometric sensor. The biometric sensor may be integrated in a button. The sensor may be a tactile button, a capacitive button, or fingerprint reader. For example, the controller may identify the first user based on a fingerprint sensed at the biometric sensor corresponding to the first user. Then, the controller may be configured to detect an input at the button that may trigger the biometric sensor to identify a user. The input device (e.g., button) may be associated with different vehicle functions corresponding to different users who have registered a biometric feature. If the controller and sensor do not identify a registered user, the controller may be configured to perform a default function in response to input at the input device. For example, the default function may adjust the equalizer settings of the stereo system.

At 220, the controller may be configured to identify the first user using the biometric sensor. The controller may be configured to adjust the first vehicle function in response to identifying the first user. Additionally, the controller may be configured to detect an input at the button in which the button is mapped to a first vehicle function based on identifying the first user. For example, the controller may be configured to detect an input at a button on a steering wheel that is mapped to a fog light function based on identifying that the first user using the sensor mounted to the vehicle.

At 230, the controller may be configured to identify the second user using the biometric sensor. The controller may be configured to adjust the second vehicle function in response to identifying the second user. Additionally, the controller may be configured to detect an input at the button in which the button is mapped to a second vehicle function based on identifying the second user. For example, the controller may be configured to detect an input (e.g., force) at an input device (e.g., lever) mounted on a door panel that is mapped to a power seating configuration based on identifying that the second user using the sensor mounted to the vehicle. In some embodiments, the sensor mounted to the vehicle is the same device as the input device. In some embodiments, the controller may be configured to perform a default function in response to the controller and sensor not identifying a registered user.

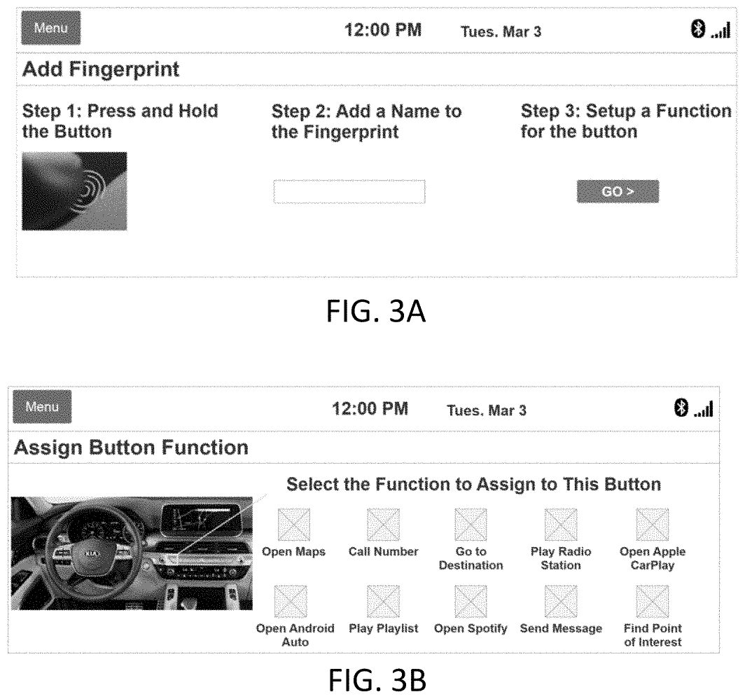

FIG. 3A and FIG. 3B depict an example of a user interface for assigning an input device to a first vehicle function based on identifying a user. The controller may be communicatively coupled to the user interface. The user interface may be integrated into the console of a vehicle. The controller and the user interface may be configured to assign a first vehicle function to an input device based on identifying a user. Additionally, and/or alternatively, the user interface may be a mobile device that is communicatively coupled to the vehicle.

To assign a vehicle function to a first user, the controller may be configured to output instructions via the user interface. For example, the controller may be configured to output instructions directing the user to press and hold a finger against a sensor. In response, the controller may be configured to detect the fingerprint of a user at a sensor. Additionally, and/or alternatively, the sensor may be integrated into an input device. The controller may be configured to detect the user engaging with or otherwise manipulating the input device as the sensor detects the fingerprint of the user. For example, the controller may be configured to detect the user engaging with a capacitive button that has a fingerprint reader built into the capacitive button.

The user interface may be configured to output a prompt for the user to enter their name. The controller may be configured to store the name of the user to be associated with the fingerprint in a database. The, the controller may be configured to output vehicle functions corresponding to the fingerprint via the user interface. For example, the controller may output a user interface with a list of vehicle functions, such as opening a map, calling a number, identifying a destination, opening a music service, communicatively coupling a mobile device, activating an HVAC setting, or sending a message. The controller may be configured to detect the selection of a vehicle function at the user interface. In response to detecting the selection of a vehicle function at the user interface, the controller may be configured to assign the fingerprint to the selected vehicle functionality.

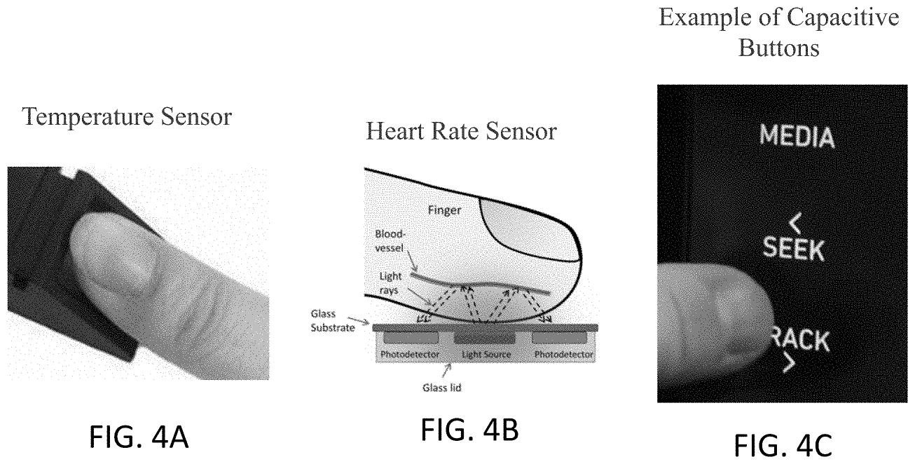

FIG. 4A depicts an example of a figure illustrating a temperature sensor. The temperature sensor may be a biometric sensor configured to determine whether a finger temperature of the user is greater than a predetermined temperature or less than a predetermined temperature. The temperature sensor may be configured to detect the temperature of the fingertip. The temperature sensor may be communicatively coupled to the controller to send data readings indicative of the temperature of the fingertip.

FIG. 4B depicts an example of a figure illustrating a heart rate sensor. The heart rate sensor may be a biometric sensor configured to determine whether a heart rate measured at a finger is higher than a predetermined heart rate or lower than a predetermined heart rate. The heart rate sensor may be configured to detect the heart rate of the user at the fingertip of the user. The temperature sensor may be communicatively coupled to the controller to send or transmit data readings indicative of the heart rate of the fingertip.

FIG. 4C depicts an example of a figure illustrating a capacitive input device. The capacitive input device may be configured to determine whether the input device is engaged by the user. The capacitive input device may be configured to detect the fingertip of the user. The capacitive input device may be communicatively coupled to the controller to send signals indicative of the input device being engaged by the user. In exemplary embodiments, the capacitive input device may be integrated into a dome light, which may send a signal indicative of how long the dome lights are to remain on.

Other types of input devices may include a lever, a switch, a touchpad, a joystick, a touchscreen, a microphone, a keyboard, a mouse, a pointing device, a brake pedal, and/or the like. In some embodiments, the input device may be a fingerprint reader behind a touchscreen in the head console. In some embodiments, the input device may be a spring-based button in a start/stop ignition configured to send signals indicative of the user identity along with a signal to turn on the engine. The input devices may be communicative coupled to the controller to send signals corresponding to the input device with which the user is engaged.

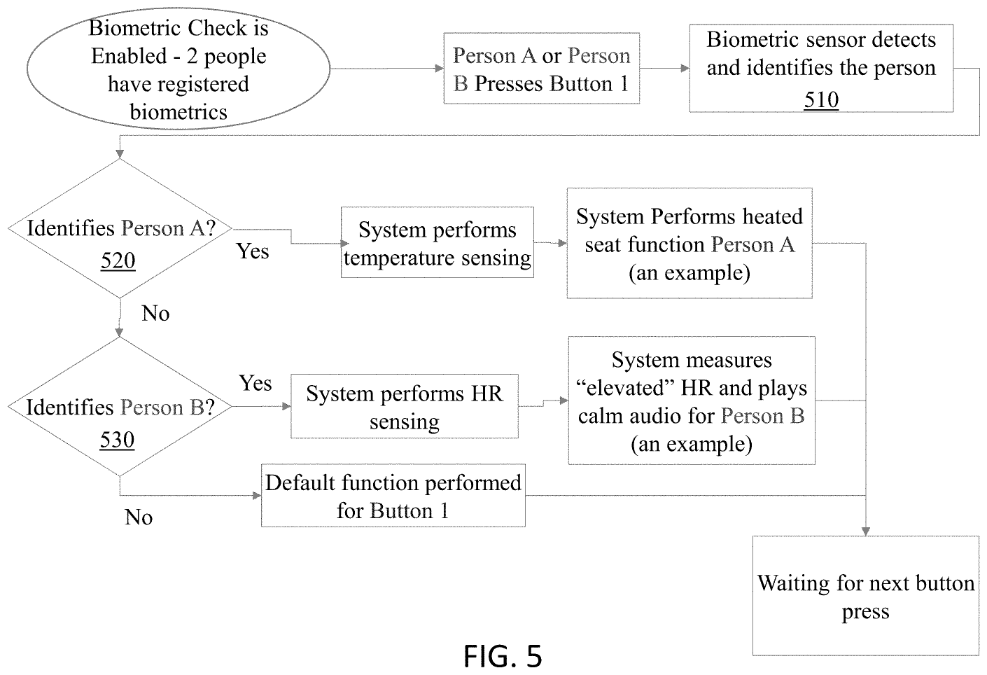

FIG. 5 depicts an example of another flowchart for adjusting a first vehicle function or a second vehicle function at a vehicle based on user identification and at least one of a temperature sensor, a heart rate sensor, and a capacitive input device. A controller may be configured to determine user identification and then adjust the vehicle function based on the user identification and input at an input device. The controller may be communicatively coupled to a sensor, a button, a temperature sensor, a heart rate monitor, and a capacitive button. The controller may be configured to detect data readings from the sensor, the input device, the button, the temperature sensor, the heart rate monitor, and the capacitive button.

At 510, the controller may be configured to receive a data reading to identify a person based on the biometric sensor. The biometric sensor may be integrated in a button, a lever, a switch, or any other input device. The biometric sensor may be a tactile button, a capacitive button, a fingerprint reader, an iris scanner, a facial recognition system, or a voice recognizer, but the present disclosure is not limited thereto. For example, the controller may be configured to identify the first user based on a fingerprint sensed by the biometric sensor corresponding to the first user. Then, the controller may be configured to detect an input at the button that may trigger the biometric sensor to identify a user. The input device (e.g., button) may be associated with different vehicle functions corresponding to different users who have registered a biometric feature. If the controller and sensor do not identify a registered user, the controller may be configured to perform a default function in response to input at the input device.

At 520, the controller may be configured to identify the first user using the biometric sensor. The controller may be configured to adjust the first vehicle function in response to identifying the first user and a data reading from an additional sensor. The additional sensor may be a biometric sensor and measure a health status, biometric, or health data of the user. For example, the controller may be configured to detect a data reading from a finger temperature reading greater than a predetermined temperature. In response, the controller may be configured to operate the steering wheel temperature to be decreased based on the finger temperature reading greater than a predetermined temperature. In another example, the controller may be configured to detect a data reading from a heart rate reading greater than a predetermined temperature heart rate. In response, the controller may be configured to operate the vehicle to diffuse a calming fragrance or select calming music based on the heart rate reading being greater than the predetermined heart rate.

At 530, the controller may be configured to identify the second user using the biometric sensor. The controller may be configured to adjust the second vehicle function in response to identifying the second user and a data reading from an additional sensor. The additional sensor may be a biometric sensor and measure a health status, biometric, or health data of the user. For example, the controller may be configured to detect a data reading from a finger temperature reading lower than a predetermined temperature. In response, the controller may be configured to operate the steering wheel temperature to be increased based on the finger temperature reading lower than a predetermined temperature.

In another example, the controller may be configured to detect a data reading from a heart rate reading less than a predetermined temperature heart rate. In response, the controller may be configured to operate the vehicle to alert passengers in the vehicle and contact emergency services based on the heart rate reading being less than the predetermined heart rate. The controller may also be configured to operate the vehicle to heat or cool seats, enter an autonomous driving mode, or activate the climate control system in response to the heart rate or the temperature being greater or less than a predetermined heart rate or temperature. In some embodiments, the controller may be configured to perform a default function in response to no user being identified using the sensor.

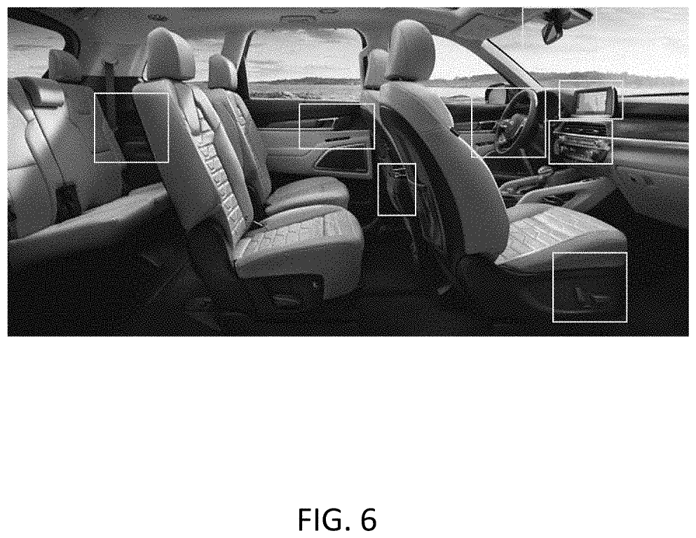

FIG. 6 depicts an example of a figure illustrating the various places for the input device in a vehicle. The input device may be mounted to the steering wheel, the rear-view mirror, the console, the user interface in the console, the power seating interface, the door panel, the rear portion of a dividing console, or a vehicle side wall. Similarly, the sensor may be mounted to the steering wheel, the rear-view mirror, the console, the user interface in the console, the power seating interface, the door panel, the rear portion of a dividing console, or a vehicle side wall.

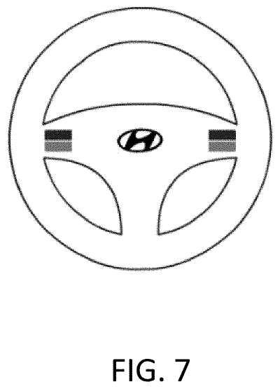

FIG. 7 depicts an example of a figure illustrating the various places for the input device on a steering wheel. The input device may be mounted on a surface facing towards the vehicle seat. The input device may be mounted on a right-hand portion or a left-hand portion of the steering wheel. The input device may be mounted on an outer edge of the steering wheel. Similarly, the sensor may be mounted on a surface facing towards the vehicle seat. The sensor may be mounted on a right-hand portion or a left-hand portion of the steering wheel. The sensor may be mounted on an outer edge of the steering wheel.

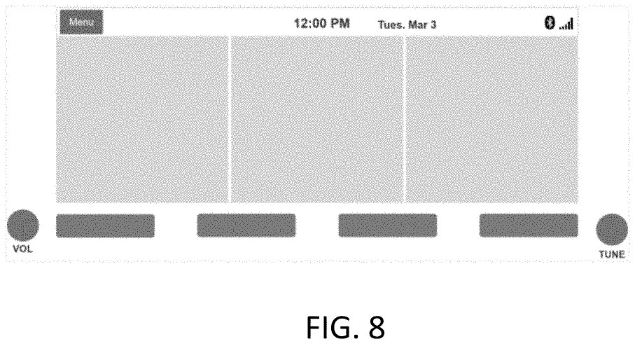

FIG. 8 depicts an example of a figure illustrating the various places for the input device in a console. The input device may be mounted as a button along the bottom edge of the console. The input device may be mounted as a switch along an edge of the console. Additionally, the input device may be integrated into the user interface presented to the user at the console. Similarly, the sensor may be mounted as a button along the bottom edge of the console. The sensor may be mounted as a switch along an edge of the console. The sensor may be integrated into the user interface presented to the user at the console.

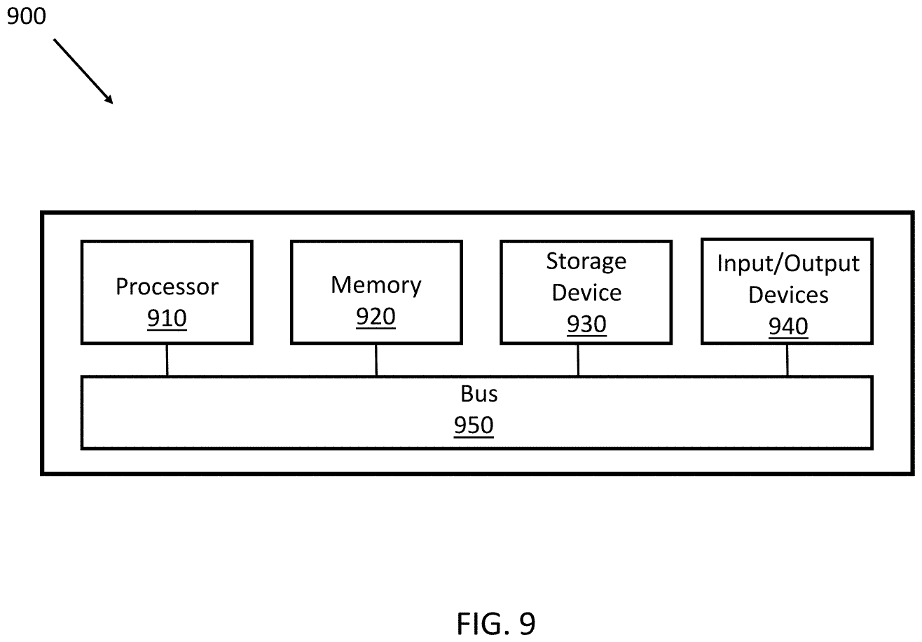

FIG. 9 depicts a block diagram illustrating a computing system 900 consistent with implementations of the current subject matter. Referring to FIGS. 1-9, the computing system 900 may be used for multi-function devices for vehicles. For example, the computing system 900 may implement a user equipment, a personal computer, or a mobile device.

As shown in FIG. 9, the computing system 900 may include a processor 910, a memory 920, a storage device 930, and an input/output device 940. The processor 910, the memory 920, the storage device 930, and the input/output device 940 may be interconnected via a system bus 750. The processor 910 is capable of processing instructions for execution within the computing system 900. Such executed instructions may implement one or more components of, for example, multi-function input devices for vehicles. In some exemplary embodiments, the processor 910 may be a single-threaded processor. Alternately, the processor 910 may be a multi-threaded processor. The processor 910 is capable of processing instructions stored in the memory 920 and/or on the storage device 930 to display graphical information for a user interface provided via the input/output device 940.

The memory 920 is a non-transitory computer-readable medium that stores information within the computing system 900. The memory 920 may be configured to store data structures representing configuration object databases, for example. The storage device 930 is capable of providing persistent storage for the computing system 900. The storage device 930 may be a floppy disk device, a hard disk device, an optical disk device, or a tape device, or other suitable persistent storage means. The input/output device 940 provides input/output operations for the computing system 900. In some exemplary embodiments, the input/output device 940 includes a keyboard and/or pointing device. In various implementations, the input/output device 940 includes a display unit for displaying graphical user interfaces.

According to some exemplary embodiments, the input/output device 940 may provide input/output operations for a network device. For example, the input/output device 940 may include Ethernet ports or other networking ports to communicate with one or more wired and/or wireless networks (e.g., a local area network (LAN), a wide area network (WAN), the Internet, a public land mobile network (PLMN), and/or the like).

In some exemplary embodiments, the computing system 900 may be used to execute various interactive computer software applications that may be used for organization, analysis and/or storage of data in various formats. Alternatively, the computing system 900 may be used to execute any type of software applications. These applications may be used to perform various functionalities, e.g., planning functionalities (e.g., generating, managing, editing of spreadsheet documents, word processing documents, and/or any other objects, etc.), computing functionalities, communications functionalities, etc. The applications may include various add-in functionalities or may be standalone computing items and/or functionalities. Upon activation within the applications, the functionalities may be used to generate the user interface provided via the input/output device 940. The user interface may be generated and presented to a user by the computing system 900 (e.g., on a computer screen monitor, etc.).

The technical advantages presented herein may result in an adjusting different vehicle functions associated with an input device based on identifying a vehicle occupant. Different vehicle occupants may have various preferences for interfacing with the vehicle to create a more convenient and familiar user experience. Since vehicles may have multiple vehicle occupants at different times, vehicle occupants may be unfamiliar with the preferences of other vehicle occupants and may experience frustration learning the different vehicle functions customized for another vehicle occupant. The present embodiments enable multiple vehicle occupants to customize the same input devices to have different functionalities based on their preferences. This eliminates user frustration in learning and experimenting with input devices to determine their functionality. Even better, vehicle occupants can adapt previously customized input devices in the vehicle to satisfy their own preferences. Additionally, the vehicle can use biometric sensors to measure a vehicle occupant's wellbeing and adapt the vehicle (e.g., adjust temperature, play soothing music) to provide a safer driving experience.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

What is claimed is:

1. A system for executing vehicle functions, comprising:
a sensor mounted on a vehicle;
an input device communicatively coupled to the sensor, the input device configured to adjust a plurality of vehicle functions based on user identification;
a controller communicatively coupled to the sensor and the input device, the controller configured to:
detect a first user input received at the input device;
identify a first user based on a first signal received from the sensor, the first signal corresponding to a first vehicle function;
in response to detecting the first user input received at the input device, adjust the first vehicle function corresponding to the first signal;
detect a second user input received at the input device;
identify a second user based on a second signal received from the sensor, the second signal corresponding to a second vehicle function; and
in response to detecting the second user input received at the input device, adjust the second vehicle function corresponding to the second signal,
wherein the first vehicle function and the second vehicle function perform different vehicle functions.

2. The system of claim 1, wherein the sensor is a fingerprint reader and the sensor is integrated into the input device.

3. The system of claim 1, wherein the first user maps the input device to the first vehicle function, the second user maps the input device to the second vehicle function, and the input device is at least one of a tactile button and a capacitive button.

4. The system of claim 1, wherein adjusting the first vehicle function is based on the first user inputting the first user input at the input device and wherein adjusting the second vehicle function is based on the second user inputting the second user input at the input device.

5. The system of claim 1, wherein the controller is further configured to:
identify, based on the sensor, the first user in response to detecting the first user input at the input device; and
identify, based on the sensor, the second user in response to detecting the second user input at the input device.

6. The system of claim 1, wherein the input device and the sensor are integrated into at least one of a steering wheel and a console of the vehicle.

7. The system of claim 1, wherein the first vehicle function and the second vehicle function are associated with a subset of the plurality of vehicle functions.

8. The system of claim 1, further comprising:
an additional sensor;
wherein the controller is further configured to:
adjust the first vehicle function based on a first data reading from the additional sensor; and
adjust the second vehicle function based on a second data reading from the additional sensor,
wherein the first data reading corresponds to a first user biometric and the second data reading corresponds to a second user biometric.

9. The system of claim 8, wherein the additional sensor is a temperature sensor, the first data reading is a temperature reading greater than a predetermined temperature and the second data reading is the temperature reading less than the predetermined temperature.

10. The system of claim 9, wherein adjusting the first vehicle function at the vehicle includes at least one of decreasing a steering wheel temperature and decreasing an air temperature in the vehicle based on the temperature reading being greater than the predetermined temperature, and wherein adjusting the second vehicle function is at least one of increasing the steering wheel temperature and increasing the air temperature in the vehicle based on the temperature reading being less than the predetermined temperature.

11. The system of claim 8, wherein the additional sensor is a heart rate sensor, the first data reading is a heart rate reading greater than a predetermined heart rate and the second data reading is the heart rate reading less than the predetermined heart rate.

12. The system of claim 11, wherein adjusting the first vehicle function includes at least one of diffusing a calming fragrance and selecting calming music based on the heart rate reading being greater than the predetermined rate, and wherein adjusting the second vehicle function includes at least one of alerting passengers in the vehicle and contacting emergency services based on the heart rate reading being less than the predetermined rate.

13. A non-transitory computer-readable storage medium storing instructions that, when executed by a processor, cause the processor to perform operations comprising:
detect a first user input received at a input device;
identify a first user based on a first signal received from a sensor, the first signal corresponding to a first vehicle function;
in response to detecting the first user input received at the input device, adjust the first vehicle function corresponding to the first signal;
detect a second user input received at the input device;
identify a second user based on a second signal received from the sensor, the second signal corresponding to a second vehicle function; and
in response to detecting the second user input received at the input device, adjust the second vehicle function corresponding to the second signal,
wherein the first vehicle function and the second vehicle function perform different vehicle functions.

14. The non-transitory computer-readable storage medium of claim 13, wherein the sensor is a fingerprint reader and the sensor is integrated into the input device.

15. The non-transitory computer-readable storage medium of claim 13, wherein the first user maps the input device to the first vehicle function, the second user maps the input device to the second vehicle function, and the input device is at least one of a tactile button and a capacitive button.

16. The non-transitory computer-readable storage medium of claim 13, wherein adjusting the first vehicle function is based on the first user inputting the first user input at the input device and wherein adjusting the second vehicle function is based on the second user inputting the second user input at the input device.

17. The non-transitory computer-readable storage medium of claim 13, wherein the processor is further configured to:
identify, based on the sensor, the first user in response to detecting the first user input at the input device; and
identify, based on the sensor, the second user in response to detecting the second user input at the input device.

18. The non-transitory computer-readable storage medium of claim 13, wherein the input device and the sensor are integrated into at least one of a steering wheel and a console of a vehicle.

19. A method comprising:
detect a first user input received at a input device;
identify a first user based on a first signal received from a sensor, the first signal corresponding to a first vehicle function;
in response to detecting the first user input received at the input device, adjust the first vehicle function corresponding to the first signal;
detect a second user input received at the input device;
identify a second user based on a second signal received from the sensor, the second signal corresponding to a second vehicle function; and
in response to detecting the second user input received at the input device, adjust the second vehicle function corresponding to the second signal,
wherein the first vehicle function and the second vehicle function perform different vehicle functions.

20. The method of claim 19, wherein the sensor is a fingerprint reader and the sensor is integrated into the input device.
